(19) United States
(12) Patent Application Publication
(10) Pub. No.: US 2004/0175708 A1
(43) Pub. Date: Sep. 9, 2004

(54) Uses of a miniature device for separating and isolating biological objects and methods used

(76) Inventors: Patrice Caillat, Echirolles (FR); Alexandra Fuchs, Saint-Egreve (FR); Frederic Revol-Cavalier, Seyssins (FR); Daniel Dupret, Sinsans-Calvisson (FR); Fabrice Lefevre, Nimes (FR)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/476,029

(22) PCT Filed: Apr. 26, 2002
(86) PCT No.: PCT/FR02/01459

(30) Foreign Application Priority Data
Apr. 27, 2001 (FR) 01/05706

(51) Int. Cl. C12Q 1/68; G01N 33/53; C12M 1/34

(52) U.S. Cl. 435/6; 435/7.1; 435/287.2; 205/777.5

(57) ABSTRACT

The invention concerns the use of a miniature device for separating and isolating biological objects for various applications in the field of molecular biology, methods for making DNA or protein chips in particular, and the DNA or protein chips obtained by using said methods.

The present invention relates to the uses of a miniature device for separating and isolating biological objects for various applications in the molecular biology field, to the methods for producing DNA chips or protein chips in particular, and also to the DNA chips or protein chips obtained using these methods.

Biology, and in particular genomics, is currently experiencing a revolution in the manner in which the data from the analyses carried out within this field are generated and processed. While more and more genetic sequence data are available by virtue of the great projects for sequencing organisms, the players in biology, in medicine and in the pharmaceutical industry are seeking to integrate all these data into large-scale and multiparametric analyses.

The world of microtechnology, in particular that of microsystems, has the means to satisfy this demand by virtue of its acquired abilities in miniaturization, in surface functionalization, in microfluidics and in large-scale, low-cost production techniques.

Currently, the coming together of these two worlds has produced tools for multiparametric analysis known as DNA chips. These tools are dedicated to the analysis of biological macromolecules (proteins and mainly DNA).

A large number of research studies exist around the world, in very varied fields, which integrate, via microtechnology, biological protocols in increasingly small sizes.

For example, microtitration plates have gone from a standard 96-well format to a 384-well and then a 1536-well format with the progress in robotics. The use of these increasingly miniaturized microtechniques makes it possible to decrease the volumes of reagents used and thus to reduce the costs of analysis.

In the particular case of DNA chips, the analytical principle consists in arranging nucleic acid probes in an X-Y matrix at increasingly small pitches, of the order of 20 μm.

Similarly, the step for treating the biological samples is subjected to the size reduction process with the increasingly common integration of polymerase chain reactions (PCR) into DNA chips or of a cell lysis function.

Thus, it has already been proposed, in particular in U.S. Pat. No. 6,071,394, to separate and attach cells on electronic chips by dielectrophoresis, the cells in suspension in a suitable buffer being separated as a function of their dielectric properties. However, this technique does not make it possible to separate and isolate single biological objects for individualized analyses.

The current tendency, which involves reducing the volumes of reagents used, results in the use of analyte assays on biological reagents attached to solid surfaces, so as to gradually abandon the use of assays in tubes with homogeneous solutions.

Thus, various solutions for attaching biological molecules of interest to various materials, such as glass, plastic or metal, have already been proposed. For example, for attaching nucleic acid probes to a substrate, three main approaches are currently known:

the use of a substrate made of glass covered with poly-L-lysine, which is a polymer exhibiting affinity with nucleic acid probes (Schena M. et al., Science, 1995, 270 (5235), 467-470),

the use of glass covered with a functionalized silane, the nucleic acid probe then bearing a complementary function so as to form a covalent bond with the silane (O'Donnell M. J. et al., Anal. Chem., 1997, 69, 2438-2443),

the use of metal electrodes, made of gold for example, allowing copolymerization of a simple monomer and of a monomer bearing the nucleic acid probe.

Similarly, in the proteomic field, the attachment of peptides via conducting polymers bearing pyrrole functions (polypyrroles) has in particular been reported (Livache T. et al., Biosensors & Bioelectronics, 1998, 13, 629-634).

It is also possible to attach living biological objects, such as bacteria or eukaryotic cells, to solid substrates by various techniques:

grafting of antibodies specific for the cell to be attached, onto a solid substrate; antibodies against the microorganisms commonly used in molecular biology (bacteria, yeast) in fact exist, such as anti-E. coli 1434 antibodies sold by the company Fitzgerald Industries International, Inc., and also antibodies against surface receptors (CD receptors) of higher eukaryotic lymphoid cells,

grafting of peptides specific for certain cell types, onto the support (Holland J. et al., Biomaterials, 1996, 17, 2147-2156),

nonspecific functionalization of the support with polymers which allow cell adhesion (Aframian D. J. et al., Tissue Eng., 2000, 6 (3), 209-216).

These techniques are advantageous in so far as they generate the formation of specific bonds between the biological object to be attached and the support; however, they require many steps of preparation and isolation before they can finally be attached individually to the support.

Furthermore, methods for identifying polynucleotide sequences and/or the corresponding proteins from a sample of nucleic acids have already been proposed, in particular in international applications WO 00/09747, WO 00/34512 and 00/34514. However, these methods require the individual preparation of DNA fragments and, consequently, many pipetting operations.

The inventors therefore gave themselves the aim of providing a miniature device for separating and isolating biological objects, making it possible to perform various types of application in the molecular biology field while at the same time conserving a matricial approach with a large number of points in which each point contains one, and only one, type or category of biological object, but in which the prior operations of preparation, separation or isolation can be avoided or reduced, thus considerably decreasing the number of manipulations and of pipetting operations, and the cost.

A subject of the present invention is therefore the use of a miniature device for separating and/or isolating biological objects, comprising at least one first electrode integrated into the device and at least one second electrode integrated into or external to the device, consisting of a structure provided with a matrix of reaction microcuvettes, each microcuvette comprising a bottom constituting a reception zone, characterized in that said bottom is devoid of holes and in that the maximum surface area of said bottom of each microcuvette is defined so as to isolate a single biological object, said structure being connected to a feed circuit so as to create a potential difference between said first electrode and said second electrode, for applications related to molecular biology, in particular for producing nucleic acid chips (DNA chips), producing protein chips, sorting genomic libraries, analyzing transcript libraries, measuring a variation in the activity of a functional protein, effecting antivirograms, and for protein screening or pharmaceutical screening.

By virtue of the device used in accordance with the invention, in which each microreservoir has at least one microcuvette, it is now possible to attach just one biological object per microcuvette, given that the surface of attachment of the biological object to be attached covers the reception zone, each microcuvette therefore containing only one type of biological object selected.

Consequently, according to the invention, the zone for attachment of the biological object to be attached or the zone to be covered by the biological object of the bottom of the microcuvette has either a surface area substantially identical to the surface area of the reception zone, or a surface area greater than the surface area of the reception zone.

According to the invention, the maximum surface area of the bottom of each microcuvette is preferably less than or equal to twice the smallest surface area of the biological object to be isolated. In a preferred embodiment of the invention, the surface area of said bottom is less than or equal to the smallest surface area of the biological object to be isolated.

More particularly, this surface area is generally between 1 μm² and 400 μm², and more preferably between 1 and 50 μm².

The biological objects selected and attached can then be treated collectively so as to implement applications related to molecular biology comprising in particular the elimination of the unattached biological objects, the separation and/or isolation of recombinant biological objects, the release of the genetic material from the biological objects, for example by chemical or electrical lysis, a step for amplification of a part of or of the genetic material that they contain.

In the case of the use of the device in accordance with the invention for separating and/or isolating recombinant biological objects, use is made, before the step for attaching the biological objects, of one or the combination of these two embodiments:

a cloning vector for transforming the biological objects, bearing a gene which encodes a specific protein which will be presented at the surface of the biological object (by fusion with the beginning of the Ipp gene for example) such that the attachment of the biological objects will take place via an interaction between this protein and a reagent as defined in the device in accordance with the invention (small molecule or specific antibody). The nontransformed biological objects will not then be attached to the miniature device in accordance with the invention;

the cloning of a DNA fragment into the cloning vector, said fragment preventing the expression of a gene encoding a protein which is toxic for the recombinant biological object.

For the purpose of the present invention, the expression “applications related to molecular biology” is intended to mean applications making it possible to obtain RNAs, DNAs or proteins on the device in accordance with the invention, in particular chemical or electrical electroporation, microinjection by microcapillary, lysis of biological objects, amplification of nucleic acids using, for example, reactions of the PCR (polymerase chain reaction) or NASBA (nucleic acid sequence-based amplification) type, rolling circle nucleic acid replication, transcription and translation of genes, etc. These applications may also correspond to reactions for transcribing DNA to messenger RNA and to reactions for translating messenger RNA to protein. They may also concern other concepts, such as recombinant protein expression, sorting a genomic library or a cell culture according to its nucleic acid or protein composition, screening (recombinant protein, enzymatic or pharmaceutical screening), in particular in the search for protein or nucleotide ligands for a given target, diagnosis, applications of the type “differential display”, a nucleic acid chip, a protein chip, analysis of transcript libraries, double hybrid, measurement of a variation in the activity of a functional protein, etc.

In particular, this device can be used for screening enzymes.

The device used in accordance with the invention makes it possible to attach a single biological object of interest to the reception zone, which in fact constitutes a trap zone.

According to the invention, the matrix of reaction microcuvettes of the device can be surmounted, at least in part, by one or more layers of isolating materials and/or by an attached grid made of biocompatible plastic, so as to form a matrix of microreservoirs. The isolating materials can, for example, be chosen from isolating polymers such as polyimides and resins such as, for example, the SU-8 resins.

According to the invention, a biological object is characterized by its container and its content. The container corresponds to any element which makes it possible to compartmentalize the content. The container may, for example, be the wall of a bacterial cell, the envelope of a virus, the membrane of a cell, a double lipid layer, micelles, a phospholipid bilayer with intrinsic proteins crossing through it, etc. The content corresponds to the biological material isolated in a compartment, namely the container. The content may, for example, correspond to nucleic acids, proteins, ribosomes, membrane vesicles, or to a complex mixture thereof.

By way of example of a biological object, mention may be made of any cell, which may or may not be healthy, whether it is prokaryotic or eukaryotic, viruses, liposomes, etc.

By way of example of a cell, mention may be made of bacteria, yeast, fungi, microalgae and also cells of plant, animal and human origin.

By way of example of a virus, mention may be made of the HIV virus, bacteriophages, etc.

The internal or external second electrode of this device also allows the subsequent attachment of one or more elements derived from the pre-attached biological object of interest, these derivatives including the products derived from lysis of the biological objects, and/or from treating them by an amplification method such as, for example, localized PCR or any other biological, chemical or electrical treatment. When these derivatives correspond to nucleic acids, reference is made to nucleic acid chips or DNA chips. When these derivatives correspond to proteins, reference is made to protein chips.

The size of the microreservoirs is defined so as to treat the isolated single biological object in a minimum volume. These microreservoirs are generally between 5 and 500 μm, and preferably between 5 and 100 μm in width and/or in length.

According to a particular embodiment of the invention, the miniature device used may comprise alternating conducting layers (electrodes) and layers of isolating materials.

According to one embodiment of the invention, one face of the first electrode integrated into the device may constitute the bottom of the microcuvettes.

According to another embodiment of the invention, the bottom of the microcuvettes of the device consists of a layer made of glass, plastic or silicon.

When the device used in accordance with the invention comprises an integrated second electrode, this electrode is placed on a first layer of isolating material and is located in a plane apart from the bottom of the microcuvettes.

When the device used in accordance with the invention comprises an external second electrode, this electrode may be joined to a cap or to a lid, preferably consisting of one or more layers of isolating material.

Consequently, one of the layers of isolating materials may not be an integral part of the device in accordance with the invention, but may be in the form of a removable, mounted component (cover, cap, lid) which covers at least in part said device and which optionally contains at least one electrode.

The device used in accordance with the invention may also comprise at least one third electrode integrated into the device, a second layer of isolating material being interposed between the second and the third electrode. In this case, and according to a variant of the invention, the device may comprise several second and/or third electrodes isolated from one another.

In the devices in accordance with the invention, at least one edge of one of the second and/or third electrodes and/or of one of the first and/or second layers of isolating materials may constitute at least one part of an edge of a microreservoir.

According to the invention, the first, second and third electrodes, and also the external electrode, consist of at least one metal layer, for example made of chromium, of gold or of platinum.

These metal layers are generally between 0.1 and 10 μm thick.

According to one embodiment of the invention, a reagent capable of attaching the biological object to be isolated is attached to at least one part of the reception zone of the reaction microcuvettes.

The nature of the reagent used to attach the biological objects can vary according to their nature and to the nature of the bottom of the microcuvettes.

Specifically, when the bottom of the microcuvettes consists of an electrode as described above, the reagent used is preferably chosen from conducting copolymers, such as, for example, polypyrroles, to which are attached proteins, peptides or any molecules specific to the type of biological object to be attached, such as, for example, antibodies, receptors, glycoproteins, lectins, cell adhesion molecules (CAM), laminin, fibronectin, integrins, sugars, etc.

Within the same device, the reagents for the microcuvettes can be identical or different.

The conducting copolymers are, for example, described in international application WO 94/22889.

Polypyrroles are particularly preferred according to the invention.

Among the peptides which can be attached to the monomers of the conducting copolymer, mention may in particular be made of peptides for specific binding to the biological objects to be isolated, such as the C₃b complement fragment and such as surface membrane receptors for the biological object, for instance peptides containing the arginine-glycine-aspartate (also referred to as RGD) sequence.

The specific molecules attached to the monomers of the conducting copolymer may in particular be chosen from protein A and protein G.

The specific molecules can be attached to the monomers of the conducting copolymer, and in particular to the pyrrole monomers, according to various techniques:

either the specific molecules are attached directly to the monomers of a conducting polymer, said monomers bearing —NHS or aldehyde functions capable of reacting with the primary amine functions of the specific molecule used,

or the specific molecules are attached indirectly to the monomers of a conducting polymer bearing the biotin function by means of successive streptavidin-biotin-specifc molecule chemical stacking. To produce this chemical stacking, the device used in accordance with the invention is then treated collectively so as to effect the copolymerization of the monomers of the conducting polymer bearing the biotin function, and then to treat said device with streptavidin then with a specific molecule bound to biotin, so as to obtain pyrrole-biotin-streptavidin-biotin-specific molecule copolymers.

In a first embodiment, the reagent used to attach the biological object may be specific for said object so as to allow a direct interaction: microcuvette reagent-biological object.

In a second embodiment, the reagent used is not specific for the biological object. Said object will therefore have to be pre-functionalized, for example, with specific antibodies which can react with the reagents used. The reagent attached only to the trap zone via the conducting polymer, such as, for example, protein A or protein G, will recognize the Fc fragment of the antibodies pre-attached to the biological objects to be immobilized.

When the bottom of the microcuvettes consists of a layer made of glass, plastic or silicon, the reagent used is preferably:

a polymer not specific for the type of biological object to be attached, such as, for example, poly-L-lysine, said polymer being deposited locally on the reception zones (“lift-off” technique: deposition of a photoimagable resin, localized insolation then deposition of the polymer on the resin, and then deblocking of the resin),

a protein or a peptide; in this case, the proteins and the peptides are attached to said layer made of glass, plastic or silicon covered with a layer of silane modified by —NHS or aldehyde functions, to which said reagent is attached; the proteins and the peptides used in this case being the same in nature as those described above.

According to a second embodiment of the invention, the reception zone of the reaction microcuvettes does not comprise any reagent able to attach the biological object, the isolation of which is desired.

In this case, the attachment of the biological objects is carried out directly via an electric field.

This embodiment is particularly advantageous since it avoids the prior functionalization of the devices in accordance with the invention with a reagent capable of attaching the biological object to be isolated. This embodiment is most particularly well suited to the isolation and to the attachment of bacteria.

The presence of at least one first electrode integrated into the device and of at least one second electrode integrated into or external to the device used in accordance with the invention makes it possible to carry out an identical treatment in all the microcuvettes, such as, for example, the application of an electric field for lysing the biological objects, an electric field for copolymerization or an electric field for membrane permeabilization.

In particular, these electrodes can enable the specific attachment of biological objects such as, for example, microorganisms, and then, once the microorganisms have been attached in the microcuvettes, the lysis or permeabilization of the microorganisms, and the attachment, by electrical copolymerization, of nucleic probes derived from a genomic amplification carried out directly in each of the microreservoirs, so as to obtain microreservoirs bearing a large amount of nucleic probes.

The second electrode present in all the devices used in accordance with the invention can either be pre-functionalized, for example with specific antibodies in order to extract from each biological object a specific protein, or more generally can be used to attach, by electrical chemistry, a product derived from the biological object subsequent, for example, to an amplification or transcription reaction.

The various applications for which the miniature devices described above are used may require replicas of the genetic material of the biological objects isolated to be made.

These replicas may be produced in different ways:

either the various biological objects isolated on a first device (parent biological objects) are placed in culture; a homogenous population of each biological object is then obtained in each microreservoir. The biological objects derived from the cell multiplication, also called offspring biological objects, can be recovered in a second device, for example by pipetting, so as to constitute the exact copy of the first device;

or the genetic material from each parent biological object is amplified, for example by PCR, using primers bearing a monomer of a conducting polymer. The amplification products may thus readily be recovered by virtue of an electric field and placed in a new device and then attached to an electrode of said new device;

or the genetic material from each biological object is transcribed into messenger RNAs on a first device (mother chip), and then the messenger RNAs thus obtained are transferred, by collective pipetting or by applying an electric field, into a second, new miniature device (daughter chip) and attached to an electrode of said new device.

The miniature devices described above can also be used:

for expressing inside or outside the biological objects a part of or their genetic material; this expression in particular comprises transcription and translation steps;

for detecting at least one property of the protein(s) isolated on the miniature device in accordance with the invention, by revealing said property;

in the context of the methods defined in international applications WO 00/09747, WO 00/34512, WO 00/34514 and WO 00/34513;

for analyzing transcripts, comprising a step for detecting a gene which has experienced a difference in expression;

for measuring a variation in the activity of a functional protein, characterized in that the effect of various molecules on the activity of said functional protein is tested;

for protein or pharmaceutical screening, in particular in the search for enzymes or for protein or nucleotide ligands for a given target.

The proteins produced in the course of this method can be revealed in various ways:

the substrate for the expressed protein, the specific revelation of which is desired, is introduced into the isolated biological objects via an electric shock;

the proteins produced, expressed in the course of this method, are removed from the isolated biological objects by an electric shock in the microreservoirs so that they can be specifically revealed in the presence of their substrate, which may, for example, be attached in a microreservoir;

the biological objects are lysed and the proteins expressed are revealed specifically in the presence of their substrate, which may, for example, be attached in a microreservoir.

This embodiment of the method is advantageous since it makes it possible to separate and characterize the functional proteins encoded by the nucleic acids of a crude sample of biological objects.

Recombinant proteins only can be attached to the protein chips using, before the step of attachment of the biological objects, a cloning vector to transform the biological objects, making it possible to label the recombinant protein with a universal epitope, such as, for example, labeling with several histidine amino acids.

The miniature devices used in accordance with the invention are preferably equipped with a closing means, such as, for example, a cap or a transparent film, making it possible to individually or collectively seal all the microreservoirs.

The use of devices comprising a first electrode integrated into the device and a second electrode external to said device is particularly suitable for permeabilization, for release of the genetic material or for lysis of the biological objects attached, for copolymerization of the nucleic acids or of the proteins derived from the biological objects or from the various molecular biology methods.

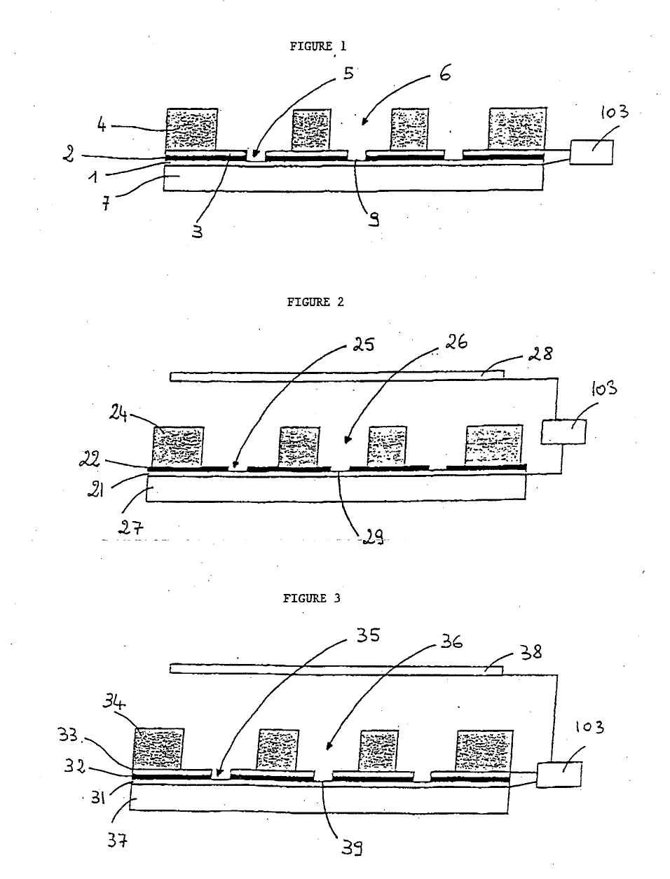

Other characteristics of the miniature devices used in accordance with the invention appear in the attached FIGS. 1 to 6, in which:

FIG. 1 represents a miniature device according to the invention, equipped with a support 7 and an electrical feed circuit 103, in which the bottom of each microcuvette 5 consists of a first electrode 1 forming a reception zone 9 to which a reagent is optionally attached, the first electrode 1 being surmounted by a first layer of isolating material 2 on which lies a second electrode 3 surmounted by a second layer of isolating material 4 forming microreservoirs 6,

FIG. 2 represents a miniature device according to the invention, equipped with a support 27 and an electrical feed circuit 103, in which the bottom of each microcuvette 25 consists of a first electrode 21 forming a reception zone 29 to which a reagent is optionally attached, the first electrode 21 being surmounted by a first layer of isolating material 22 on which lies a second layer of isolating material 24 forming microreservoirs 26, this device being equipped with an external second electrode 28,

FIG. 3 represents a miniature device according to the invention, equipped with a support 37 and an electrical feed circuit 103, in which the bottom of each microcuvette 35 consists of a first electrode 31 forming a reception zone 39 to which a reagent is optionally attached, the first electrode 31 being surmounted by a first layer of isolating material 32 on which lies a second electrode 33 surmounted by a second layer of isolating material 34 forming microreservoirs 36, this device being equipped with an external electrode 38,

FIG. 4 represents a miniature device according to the invention, which is identical to that represented in FIG. 1 except that it also comprises a removable closing means 100 which makes it possible to close each of the microreservoirs 46,

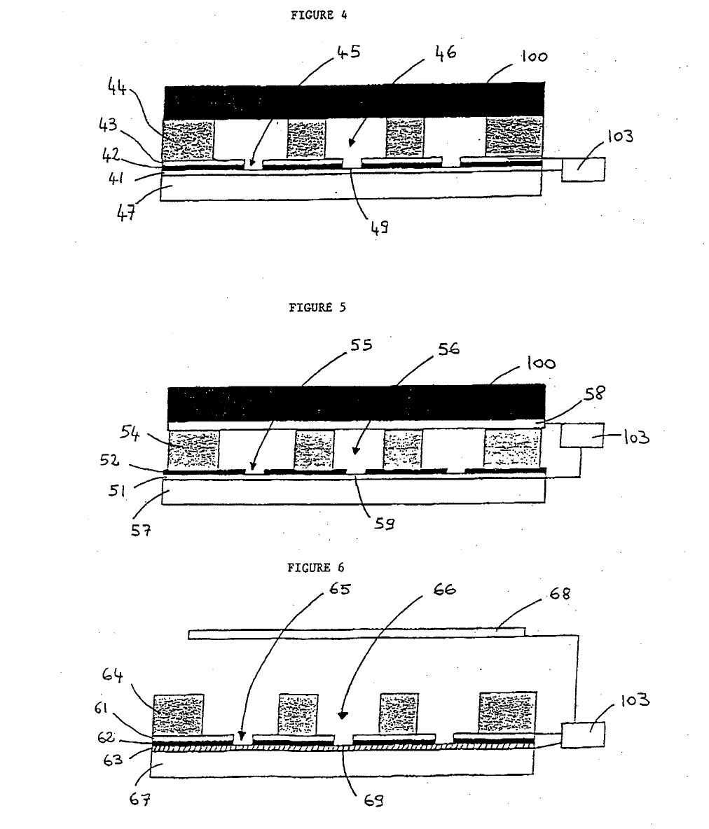

FIG. 5 represents a miniature device according to the invention, which is identical to that represented in FIG. 2 except that it also comprises a removable closing means 100 which makes it possible to close each of the microreservoirs 46, into which an external second electrode 58 is integrated,

FIG. 6 represents a miniature device according to the invention, equipped with a support 67 and an electrical feed circuit 103, in which the bottom of each microcuvette 65 consists of a layer of glass or of silicon 63 forming a reception zone 69 to which a reagent is optionally attached, said layer of glass or silicon 63 being surmounted by a first layer of isolating material 62 forming microreservoirs 66, on which layer lies a first electrode 61, itself surmounted by a second layer of isolating material 64, this device being equipped with an external second electrode 68.

It is of course understood that the devices illustrated in these figures correspond to particular embodiments of the invention and in no way constitute a limitation thereof.

The methods for producing such devices are known and are described, for example, in French patent application FR-A-2 781 886.

According to the invention, the biological objects to be isolated may be chosen from cells which may or may not be healthy, such as, for example, cells infected with viruses or malignant cells. These biological objects may therefore correspond to prokaryotic or eukaryotic cells, viruses, liposomes or microalgae. According to a particularly preferred embodiment of the invention, the biological objects to be isolated are chosen from bacteria and yeast, the bacteria preferably being derived from a library of genomic sequences.

According to a particular embodiment of the invention, these miniature devices can be used for obtaining nucleic acid chips or protein chips, or else for detecting functional proteins. These DNA chips or protein chips constitute another subject of the invention.

A subject of the invention is therefore also a method for separating and/or isolating a biological object for obtaining nucleic acid chips or protein chips or for detecting functional proteins, characterized in that it comprises at least the following steps a) and b):

a) bringing at least one miniature device as defined above into contact with a homogenized solution of biological objects, in particular with a biological cell culture solution, so as to allow the attachment of said biological objects at the bottom of the microcuvettes, on the reception zones, at a rate of at most one biological object per microcuvette,

b) removing the unattached biological objects so as to obtain a miniature device on which the biological objects to be isolated are immobilized.

According to a first embodiment of the method in accordance with the invention, the biological objects are attached via an electric field. In this case, devices in which the first electrode integrated into the device constitutes the bottom of the microcuvettes are preferably used.

According to a second embodiment of the method in accordance with the invention, the biological objects are attached via a reagent attached to at least one part of the bottom of the reaction microcuvettes. In this case, the bottom of the microcuvettes can equally consist of a first electrode or of a layer made of glass, plastic or silicon.

The method in accordance with the invention may comprise a step which is a step preliminary to step a), consisting in transforming the biological objects with a cloning vector bearing a gene which encodes a specific protein which will be presented at the surface of the biological objects such that the attachment of the biological objects will take place via an interaction between this protein and a reagent specific for said protein and as defined above, and/or by cloning a DNA fragment into a vector, said fragment preventing the expression of a gene encoding a protein which is toxic for the recombinant biological object.

According to a preferred embodiment of the invention, use is made of a device comprising microreservoirs and this method comprises a step during which the genetic material of the biological objects is released into said microreservoirs by any technique known to those skilled in the art (lysis, electric shock, etc.).

The devices used during this method also preferably comprise an internal or external second electrode suitable for permeabilization of the attached biological objects.

When this method is used for obtaining nucleic acid chips, it then comprises a step during which the genetic material of the isolated objects is amplified so as to obtain amplified sequences. This amplification step is generally followed by a step of attachment by electropolymerization of the amplified sequences on a second electrode of the device used.

When this method is used for obtaining protein chips or for detecting functional proteins, it preferably comprises, after step b), a step for transcription and translation of the genetic material of the isolated biological objects into proteins. The proteins thus expressed can be attached, as described above, to a second electrode of the device used.

This method can in particular be used for obtaining recombinant protein chips and, in this case, before step a), the method then comprises a step for transforming the biological objects with a cloning vector in order to enable the recombinant proteins to be labeled with a universal epitope.

When this method is used for detecting functional proteins, it then comprises, after the translation step, a step for revealing at least one property of the isolated proteins.

A measurement of the variation in the activity of the functional proteins thus isolated can also be carried out according to a method consisting in testing the effect of various molecules on the activity of said functional proteins.

According to a particular embodiment of the invention, the method for producing a nucleic acid chip (DNA chip) is characterized in that it comprises the following steps:

a) bringing at least one miniature device as defined above into contact with a homogenized solution of biological objects, in particular with a solution of a culture of biological cells, so as to allow the attachment of said biological objects at the bottom of the microcuvettes, on the reception zones, at a rate of at most one biological object per microcuvette,

b) removing the unattached biological objects so as to obtain a miniature device on which the biological objects to be isolated are immobilized,

c) introducing into the microreservoirs a common reaction mixture containing all the reagents required for a genomic amplification, said reaction mixture comprising in particular at least one primer functionalized with pyrrole groups,

d) lysing or permeabilizing the biological objects attached at the bottom of the microcuvettes so as to bring the genetic material which they contain into contact with the reagents required to amplify the sequences,

e) attaching the amplified sequences to a second electrode so as to obtain a nucleic acid chip.

In order to obtain these DNA chips, it is possible to use the devices such as those illustrated in FIGS. 1, 3 and 6. In this case, use is preferably made of devices comprising microcuvettes the bottom of which has a surface area of approximately 1 μm², the cell culture then preferably being a bacterial library of nucleotide sequences.

The method in accordance with the invention requires neither micropipetting nor delicate positioning of the objects to be isolated.

The advantage of this method is that all the probes of the chip are produced collectively, the nature of the probes and their degree of representation being directly related to the diversity of the starting culture.

For example, the starting cells (bacteria, yeast) may be derived from the transformation of host cells with a library of recombinant vectors bearing nucleotide sequences (genomic sequence, cDNA sequence, differential sequence, etc.). The probes are produced by amplification of the nucleotide sequences contained in the recombinant vectors by virtue of primers included in the vector. The chip is then used as a matrix for plating out a library of clones. The chip is random in that the exact nature of each probe in each microcuvette is not known. This is the principle of cloning.

In fact, the cloning of bacteria or of yeast is usually carried out by plating cells out on a solid nutritive medium. The cells are thus spatially individualized. After many divisions (more than 20 hours of culturing at least), the individualized cell has formed a colony of cells which are all identical. The multiplication of the cells has thus indirectly induced the amplification of the sequence present in the recombinant vector.

The difficulty in this conventional cloning method is being able to evenly plate out colonies which are clearly independent, a maximum of 20-30 bateria/cm², so as to then be able to isolate the colony of interest.

It is then necessary to place this colony of interest back in culture, which requires a further few hours of additional culturing.

On the other hand, according to the method in accordance with the invention, the equivalent of 5000 clones/cm² can rapidly be realized, with ordered plating out. The amplification can be carried out in one hour and the grafting with polypyrrole is instantaneous.

The difficulties in culturing certain microorganisms, in transferring onto a membrane and in attaching DNA are thus avoided.

The DNA chips in accordance with the invention, can, for example, be used for the large-scale screening of a gene library.

They in particular make it possible:

to find the complete sequence of a gene using a known fragment which will be labeled and hybridized on the chip, the labeled (positive) microreservoir(s) containing the sequence of the gene being sought;

to find homologous genes by relatively nonstringent hybridization (inter-species homologous genes or genes from the same family), using the sequence of a starting gene which will be labeled and hybridized on the chip, the positive microreservoir(s) containing the sequence of homologous genes;

to determine the degree of representation of a particular gene in the library using the gene in question as a probe and dividing the number of positive microreservoirs by the total number of microreservoirs in order to evaluate the quality of the library and to evaluate the criteria for the degree of representation of the genes according to their amount and their size;

to evaluate the quality of a standardized library (all the genes being represented the same number of times), the quality of a subtraction library (no cross-hybridization on the same microreservoirs).

The DNA chips in accordance with the invention can also be used for screening DNA as a function of a target protein. In this case, the probes attached to the chip are a panel of promoters. The sequence to which a given trans-regulating target protein attaches is sought. This protein is pre-labeled and is incubated in each microreservoir of the chip. The positive microreservoirs contain the sequence of the promoter specific for the protein.

The devices in accordance with the invention, and in particular the devices illustrated in FIGS. 1 and 3 in which the second electrode has been functionalized with a specific antibody against a universal epitope (anti-HA, anti-myc or anti-polyhystidine antibodies), can also be used for producing protein chips.

These protein chips constitute another subject of the invention.

A subject of the invention is therefore also a method for producing a protein chip, characterized in that it comprises the following steps:

a) bringing at least one miniature device as defined above into contact with a solution of a culture of biological cells transformed with a recombinant vector bearing a gene which makes it possible to subsequently sort the recombinant clones, said vector also enabling the recombinant protein to be labeled with a universal epitope (of the polyhistidine type for example), so as to allow the attachment of said cells at the bottom of the microcuvettes, on the reception zones, at a rate of at most one cell per microcuvette,

b) removing the unattached cells so as to obtain a miniature device on which the cells to be isolated are immobilized,

c) introducing into the microreservoirs reagents which induce the transcription and translation of the genetic material so as to allow the synthesis of the corresponding proteins,

d) releasing the recombinant proteins expressed, for example by lysing the biological objects attached at the bottom of the microcuvettes,

e) attaching the proteins bearing the universal epitope to a second electrode internal to the device, said electrode having been pre-functionalized, for example with anti-universal epitope antibodies,

f) optionally removing, by washing, the other constituents of the biological objects, so as to obtain a protein chip.

Only the recombinant proteins are retained on this chip.

The random protein chip thus obtained can be used, for example, for carrying out biochemical assays or protein/protein recognition assays with a labeled target protein for which it is desired to identify, in the library, the protein partners with which it can interact, such as for example the double-hybrid assay, or else protein/DNA protein/sugar recognition assays, etc.

The production of the protein chips can also be carried out without prior sorting of the library.

According to a particular embodiment, and when the devices illustrated in FIGS. 1 and 3 are, for example, used, all the reagents can be attached via pyrrole groups. Pyrrole monomers bearing —NHS functions are then synthesized, which monomers are coupled via NHS—NH₂ bonds to various antibodies, for example, to an antibody directed against the universal epitope, another antibody being specific for the cell to be immobilized. A monomer of pyrrole-antibody 1 (directed against the cell to be immobilized) and a monomer of pyrrole-antibody 2 (directed against the universal epitope) are thus, for example, obtained. The device in accordance with the invention is then immersed in a solution containing a pyrrole/pyrrole-antibody 1 mixture and the first electrode is activated in order to functionalize the bottom of the microcuvettes.

After rinsing, the device in accordance with the invention is then immersed in a solution containing a pyrrole/pyrrole-antibody 2 mixture and the second electrode is activated in order to functionalize it.

The device thus functionalized is then soaked in a solution of bacteria. By virtue of recognition and steric hindrance, a single bacterium is then attached at the bottom of each of the microcuvettes, on the first electrode.

The proteins are then translated inside the bacterium, as described above, and the bacteria are then lysed so as to release the universal epitope proteins synthesized which, if they are recognized by the antibody 2, will be attached to the second electrode.

According to another particular embodiment of this method, and when use is made of a device comprising microcuvettes the bottom of which consists, for example, of a layer of glass, such as that represented in FIG. 6, it is possible to attach the bacterium via a nonspecific polymer or via NHS silanization and attachment of an antibody 1 specific for the type of cell to be immobilized, and then the procedure as described above is carried out.

According to a variant of this method, the production of protein chips can be carried out by performing the following steps consisting in:

a) bringing at least one miniature device as defined above, and in which the bottom of the microcuvettes consists of a layer of glass, into contact with an emulsion in which each micelle contains a gene and the reaction medium required to express the gene in vitro in a compartmentalized manner, so as to allow the attachment of said micelles at the bottom of the microcuvettes, on the reception zones, at a rate of at most one micelle per microcuvette,

b) optionally removing the unattached micelles so as to obtain a miniature device on which the micelles to be isolated are immobilized,

d) lysing the micelles attached at the bottom of the microcuvettes so as to release the proteins expressed,

e) attaching the proteins to a second electrode internal to the device and which is functionalized,

f) optionally removing, by washing, the other constituents of the micelles so as to obtain a protein chip.

The preparation of this emulsion can be carried out in a known manner and as described, for example, by Tawfik and Griffiths, Nature Biotechnology, 1998, 16, 652-656.

An emulsion can also be produced in the presence of a DNA solution in such a way as to compartmentalize DNA fragments in micelles.

Each micelle is then isolated using a miniature device as described above, and then lysed so as to release, in each microreservoir, the DNA fragment(s) which may then be locally transcribed and then translated so as to study the function of the proteins thus obtained. This embodiment of the device of the invention makes it possible to separate and characterize functional proteins in a miniature device as described above.

According to yet another variant of the invention, the miniature devices in accordance with the invention can be used for producing a random protein chip while at the same time keeping the information of the DNA sequence encoding the protein, which will have been synthesized as above, in the form of a random DNA chip.

To do this, the cells of the library are immobilized as described above at the bottom of the microcuvettes of a first chip A on which a few cell divisions are carried out. A copy of the first chip A is produced in a second chip B, by transferring cells, in an ordered manner, from the microreservoirs of chip A to the corresponding microreservoirs of chip B, the microreservoirs of chip A optionally being placed facing the microreservoirs of chip B, with agitation.

Chip A is then subjected to the treatment described above for producing a random DNA chip, while chip B is subjected to the treatment described above for obtaining a corresponding random protein chip. After identification of the positive microcuvettes (either by a protein assay on chip B, or by a DNA assay on chip A), the sequence of the DNA on chip A or that of the corresponding protein on chip B is then directly obtained.

A subject of the invention is also a method for screening proteins, which may or may not be recombinant, characterized in that it comprises the following steps:

a) bringing at least one miniature device (mother chip) as defined above into contact with a homogenized solution of biological objects, in particular with a solution of a culture of microorganisms, so as to allow the attachment of said biological objects at the bottom of the microcuvettes, on the reception zones, at a rate of at most one biological object per microcuvette,

b) optionally removing the unattached biological objects so as to obtain a miniature device on which the biological objects to be isolated are immobilized,

c) introducing into the microreservoirs a common reaction mixture containing all the reagents required for a genomic amplification, said reaction mixture comprising in particular at least one primer functionalized with pyrrole groups,

d) lysing or permeabilizing the biological objects attached at the bottom of the microcuvettes so as to bring the genetic material which they contain into contact with the reagents required for an amplification and to thus allow said amplification,

e) introducing into each microreservoir a reaction mixture containing at least one RNA polymerase for transcribing the amplified sequences into messenger RNAs (transcripts),

f) introducing into each microreservoir a reaction mixture for translating the transcripts obtained in step e) into corresponding proteins,

g) testing at least one property of the proteins obtained in step f) by introducing an appropriate substrate into each microreservoir, and optionally,

h) lysing the biological objects, if this has not been carried out during step d), and then sequencing the genes contained in the microreservoirs in which a positive protein/substrate reaction has taken place.

According to this screening method, steps c) and d) can optionally be carried out simultaneously.

When the biological objects have been lysed in step d), then the method in accordance with the invention can comprise an additional step consisting in collectively attaching the amplified sequences to an electrode by electropolymerization. In this case, a step consisting of washing the microreservoirs can then be carried out in order to remove the remainder of the lysed biological objects and, optionally, the amplified sequences not attached to the electrode.

Also, when the biological objects have been lysed in step d), the screening method in accordance with the invention can comprise another additional step consisting in polarizing an electrode in order to attract the messenger RNAs synthesized in step (e) to the bottom of the microreservoirs, this polarization step optionally being followed by a step consisting of washing the microreservoirs.

The screening method in accordance with the invention requires neither micropipetting nor delicate positioning of the biological objects to be isolated.

This method in fact makes it possible to screen proteins on an ordered matrix while at the same time not requiring any individual pipetting in order to create said matrix.

It also makes it possible to limit the number of times sequencing must be carried out, since only the genes contained in the microcuvettes which have given a positive protein/substrate reaction are sequenced.

According to a particularly preferred embodiment of this method, the device used is equipped with a closing means making it possible to close the microreservoirs while the various reactions are taking place (lysis or permeabilization of the biological objects, genomic amplification, transcription of the amplified sequences into messenger RNAs, translation of the transcripts into corresponding proteins, etc.).

The reaction mixtures for transcribing the amplified sequences into messenger RNAs and translating the transcripts into proteins are those conventionally used for carrying out these operations. They are, for example, described in detail in international application WO 00/09747.

According to a preferred embodiment of the invention, steps (e) and (f) are carried out simultaneously.

According to an advantageous variant of this method, and when the biological objects have been lysed in step (d) and the amplified sequences have been attached to an electrode by electropolymerization, then the messenger RNAs obtained at the end of step (e) can be transferred, for example by collective pipetting, into a fresh miniature device (daughter chip) identical to the preceding one, in which steps (f) and (g) as defined above will take place.

This transfer makes it possible to preserve the integrity of the amplified sequences in the microreservoirs of the first device. Specifically, the reagents used during the translation step (f) contain DNases which damage the DNA of the amplified sequences, subsequently prohibiting exploitation of the information which they contain.

This transfer may in particular be carried out by means of a negative tool with metal pins in which an electric field is applied so as to attract the messenger RNAs. The latter will then be deposited in the microreservoirs of the daughter chip by depolarizing the metal pins of this tool. The messenger RNAs thus transferred onto the daughter chip may then be attracted onto an electrode of the daughter chip by applying an electric field.

The mother chip/daughter chip copy can also be produced subsequent to step (b) and after a few cell divisions in the presence of a suitable culture medium, by simply placing the miniature device used (mother chip) facing another fresh miniature device of the same type (daughter chip). The mother chip and the daughter chip then both undergo steps (c) and (d) as described above, while only the daughter chip then undergoes step (e).

When a mother chip and a daughter chip are used in accordance with the invention, devices such as that represented in FIG. 2, i.e. comprising a first electrode integrated into the device and a second electrode external to said device, can be used.

The daughter chip then collectively receives the reaction mixture containing the reagents required for translating the messenger RNAs into corresponding proteins (labeled amino acids) and vesicles carrying a reactive substrate, such as, for example, liposomes, in order to assay the activity of the synthesized proteins.

Once the protein translation reaction has taken place, a first verification of the synthesis of the proteins is carried out microreservoir by microreservoir, for example by polarized fluorescence measurement, in order to distinguish the radiation emitted as a function of the size of the molecules (labeled amino acids/proteins).

The reactive substrate for assaying the activity of the synthesized proteins is then released locally in each microreservoir by rupturing of the vesicles under the action of sonication. This substrate is then possibly modified by the action of the synthesized proteins and the modification of this substrate is detected by any appropriate means.

This method therefore makes it possible to determine which microreservoir has generated the protein responsible for the modification of the substrate assayed. It is then sufficient to sequence the amplified sequence attached in the corresponding microreservoir (positive microreservoir) of the mother chip.

The mother chip then undergoes a further cycle of genomic amplification using primers which do not contain pyrrole functions.

The content of the positive microreservoirs can then be locally pipetted and distributed into individual tubes in which conventional sequencing may be carried out.

When the miniature devices as defined above are used to analyze a transcript library, they make it possible to determine which genes undergo differential expression in response to stress, such as, for example, thermal shock, exposure to a drug, a pathological condition.

A subject of the invention is therefore also a method for analyzing a transcript library, characterized in that it comprises steps (a) and (b) described above (attachment of the biological objects to a miniature device and washing of the unattached biological objects) and also a step for detecting a gene which has experienced a difference in expression.

This method is preferably characterized in that, in a first step, the following sub-steps are carried out, consisting:

in isolating biological objects A on a first miniature device as defined above (mother chip A),

in washing the unattached biological objects A so as to obtain a device on which the biological objects A to be isolated are immobilized, then

in extracting the messenger RNAs from the biological objects A (“stressed” cells A), for example by lysing the various objects isolated;

in subjecting the cell content to treatment with DNase,

in transferring the extracted messenger RNAs into the microreservoirs of a new device (daughter chip A), for example by means of a negative tool with metal pins in which an electric field is temporarily applied so as to attract and then deposit the messenger RNAs,

in attracting the messenger RNAs onto an electrode of the daughter chip A by means of an electric field,

in synthesizing the corresponding cDNAs

in labeling the cDNAs thus synthesized using a nucleotide segment comprising at least one promoter of in vitro transcription and at least one segment which allows their specific attachment in a microreservoir of the daughter chip A.

Each labeled cDNA strand is thus attached in the microreservoirs of the daughter chip A.

These same operations can be carried out using biological objects B (cells identical to the cells A, but not stressed). In the same way, the corresponding cDNAs will be attached in microreservoirs of a daughter chip B.

In a second step, the daughter chip A is then immersed in a solution containing the nonlabeled strands of the cDNAs from cell B. If a nonlabeled strand hybridizes on its complementary strand on the daughter chip A, this makes it possible to reconstitute a functional double-stranded transcription promoter.

Carrying out an in vitro transcription step in each microreservoir, using fluorescent nucleotides, makes it possible to reveal which microreservoir contains a cDNA from cell B capable of hybridizing on a cDNA from cell A.

In parallel, the daughter chip B is immersed in a solution containing the nonlabeled strands of the cDNAs from cell A and the same revelation is then performed. It is then revealed which microreservoir contains a cDNA from cell A capable of hybridizing on a cDNA from cell B.

In a third step, the daughter chips A and B are treated in order to remove the mRNAs produced and also the nonlabeled cDNAs.

The daughter chips A and B are then immersed in a solution containing the nonlabeled strands of the cDNAs of the cells A and B, respectively, and a further revelation by transcription is performed.

The signal obtained in each microreservoir is then compared with the signal which was obtained in the preceding step, and the genetic material present in the microreservoirs negative at the end of the second step and positive at the end of the third step is recovered in order to be characterized, for example by performing a reverse transcription in order to sequence the genes which have undergone differential expression.

The miniature devices as described above can also be used for effecting antivirograms.

Antivirograms consist in defining the inhibitors of a viral enzyme essential to the multiplication cycle of a virus, thus making it possible to identify virucidal active principles, in an embodiment corresponding to the infection of cells with a single form of a virus.

A subject of the invention is therefore also a method for effecting an antivirogram, characterized in that it comprises the following steps consisting in:

isolating infected or potentially infected cells on a miniature device as defined above,

lysing said cells in order to release the gene encoding the enzyme essential for the multiplication cycle of the virus,

amplifying said gene, for example by any technique known to those skilled in the art,

attaching the amplification products to a second electrode; copolymerization polymers will, for example, be used for this purpose,

optionally removing, by washing, the other constituents of the cells so as to retain only the amplification products,

transcribing and then translating into proteins said amplification products in the microreservoirs,

revealing the activity of the proteins obtained in the presence of inhibitors which may or may not be identical, in each microreservoir, so as to reveal the inhibitors capable of inhibiting, in vitro, a viral enzyme essential to the multiplication of a virus.

This type of experiment also makes it possible to study the degree of representation of the enzyme when several isoforms of this enzyme exist.

The miniature devices as described above can also be used according to another embodiment of an antivirogram.

In this case, the cells of a library transformed with the genes encoding the various isoforms of a viral enzyme essential to the multiplication cycle of a virus are isolated by virtue of a miniature device as described above. Only the recombinant cells are isolated (either by sorting the library as described above, or by pre-selection on selective medium). The cells not retained on the device are removed by washing. The proteins encoding the viral enzyme can then be expressed and studied as previously so as to give an antivirogram of the viral enzyme studied.

Besides the above arrangements, the invention also comprises other arrangements which will emerge from the following description, which refers to examples of immobilization of bacteria on miniature devices in accordance with the invention, to an example describing the protocol for preparing a DNA chip on a device according to the invention, and also to an example of expression of a genetic material on a miniature device in accordance with the invention.

### EXAMPLE 1

### Isolation and Attachment of Bacteria on a Miniature Device Via Protein A

A solution of protein A at 0.1 mg/ml in phosphate buffer (PBS) is prepared.

Using a pipette, a drop of this protein A solution is then deposited onto a miniature device in accordance with the invention and as described in the attached FIG. 1, such that said drop covers all the microcuvettes.

On this device, each microreservoir has a diameter of 230 μm and a depth of 40 μm; the surface area of the bottom of each microcuvette is 40 μm².

An electric field is then applied, for 10 seconds, between the two electrodes of the chip: potential of +2.9 V on the electrode where the attachment of the protein A is desired, the other electrode being earthed.

When the attachment has been carried out, the device is then rinsed with a PBS solution.

In addition, a solution of PBS containing a bacteria/antibody complex is prepared.

To do this, a solution of E. coli DH5α in PBS (10⁹ bacteria/ml) is first prepared along with a solution of the corresponding anti-E. coli antibody (Dako) at 0.5 mg/ml.

These two solutions are then mixed (V/V) and left stirring at ambient temperature for 1 hour and thirty minutes in order to form the bacteria/antibody complex. The bacteria/antibody complex is then concentrated by centrifugation, the excess antibodies being eliminated by removing the supernatant. The operation is repeated three times, after re-dissolving the bacteria/antibody complex in PBS.

Using a pipette, a drop of the solution containing the bacteria/antibody complex is then deposited onto the miniature device functionalized with protein A, such that said drop covers all the microcuvettes.

The miniature device is then left to incubate for 1 hour 30 minutes at ambient temperature, in order to allow immobilization of the bacteria/antibody complex at the bottom of the microcuvettes.

At the end of the incubation, the device is then rinsed thoroughly with PBS in order to remove the bacteria/antibody complexes which have not reacted with the protein A.

A device is obtained on which E. coli bacteria are immobilized at a rate of one bacterium per microcuvette.

The miniature device in accordance with the invention thus prepared can then be used in various biological applications.

### EXAMPLE 2

### Isolation and Attachment of Bacteria on a Miniature Device Under the Action of an Electric Field

1) Attachment of the Bacteria Via Electric Field

A suspension of E. coli DH5a bacteria in deionized water in a proportion of 10⁹ bacteria/ml is prepared.

A miniature device identical to that used above in example 1 is then immersed in this bacterial suspension.

An electric field is then applied, for 10 seconds, between the two electrodes of the chip: potential of +0.9 V on the electrode where it is desired to attach the bacterium, the potential of the other electrode being fixed at −2V.

When the attachment has been carried out, the device is then rinsed with water and dried with a nitrogen blow gun.

A device is obtained, on which E. coli bacteria are immobilized at a rate of one bacterium per microcuvette.

The miniature device in accordance with the invention thus prepared can then be used in various biological applications.

### EXAMPLE 3

### Preparation of a DNA Chip by PCR Using a Miniature Device in Accordance With the Invention

This example describes the general protocol for preparing a DNA chip on a miniature device in accordance with the invention.

1) Depositing a Sense Primer on a Miniature Device in Accordance With the Invention

A 2.3⁻⁴ M solution of a sense primer modified in the 5′ position with a pyrrole group (0.77 μM) is prepared in 2.3⁻² M lithium perchlorate.

This solution is deposited onto the miniature device in accordance with the invention and as prepared above in example 2.

An electric field is then applied, for 3 seconds, between the two electrodes of the chip: potential of +2.9 V applied to the electrode where attachment of the primer is desired, the other electrode being earthed.

The miniature device is then rinsed with water and dried with a nitrogen blow gun.

2) Lysing the Bacteria

This step is carried but by heating the bacteria at a temperature of 94° C. for 2 minutes.

3) Performing the PCR

The PCR is performed using the following solution: 1 mM Tris-HCl, 5 mM KCl, 2 mM MgCl₂, 0.8 mM dNTP; antisense primer labeled with biotin in the 5′ position: 0.1 μM, 1 mg/ml BSA, Taq DNA polymerase from Roche at 0.02 units/μl and sense primer at 0.01 μM.

The chip is then immersed in oil.

The PCR is performed under the following conditions: 3 minutes at 94° C. then 30 cycles at 94° C. for 30 seconds, 60° C. for 30 seconds and 72° C. for 1 minute 30 seconds; then 72° C. for 3 minutes and, finally, 25° C. for 30 seconds. The cycles are performed in a Hybaid thermocycler.

The miniature device is then rinsed with water after the end of the PCR cycles.

The amplified DNA is fluorescently labeled with streptavidin-phycoerythrin.

The fluorescence is then visualized using a fluorescence microscope.

### EXAMPLE 4

### Expression of a Genetic Material on a Miniature Device in Accordance With the Invention

In this example, a miniature device identical to that used in examples 1 to 3 above and comprising 8100 wells was used.

E. coli bacteria containing the beta-lactamase TEM-1 bla gene were immobilized on this device according to the immobilization method described above in example 2.

The bla gene was then amplified by PCR under the conditions described above in example 3.

The PCR product thus obtained comprises (from the 5′ position to the 3′ position) a T7 promoter, a ribosome-binding site and the open reading frame of the bla gene.

A control chip, intended to be used as a control, was produced by amplifying in the same way the green fluorescent protein GFP gene.

In order to allow the expression of the proteins, these two chips were covered with a coupled in vitro transcription/translation reaction mixture.

This mixture was prepared according to the method as described by Pratt and Nevin (Pratt J. M., “Coupled transcription-translation in prokaryotic cell-free systems, Transcription and Translation: A practical approach”, Hames B. D. & Higgins S. J., JLR Press, 1984, 179-209; Nevin D. E. and Pratt J. M., “A Coupled in vitro transcription-translation system for the exclusive synthesis of polypeptides expressed from the T7 promoter, FEBS, 1991, 291, 2, 259-263).

Each chip was then covered with a cover glass slide and placed in a vacuum bell so as to allow efficient filling of each microreservoir by degassing.

The chips thus prepared were incubated in an atmosphere saturated with water (to limit evaporation of the reagents) for 90 minutes at 30° C. The cover glass slides were then removed and each chip was covered with nitrocefin (Oxoid, ref. SR112C). This reagent has the property of turning from yellow to red when it is hydrolyzed by a beta-lactamase.

The results obtained (not shown) are as follows:

On the control chip (GFP gene), the reagent remained yellow.

On the chip comprising the bla gene PCR product, the reagent turned red, indicating that the beta-lactam TEM-1 was expressed on this chip.

1. The use of a miniature device for separating and/or isolating biological objects, comprising at least one first electrode integrated into the device and at least one second electrode integrated into or external to the device, consisting of a structure provided with a matrix of reaction microcuvettes, each microcuvette comprising a bottom constituting a reception zone, characterized in that said bottom is devoid of holes and in that the maximum surface area of said bottom of each microcuvette is defined so as to isolate a single biological object, said structure being connected to a feed circuit so as to create a potential difference between said first electrode and said second electrode, for applications related to molecular biology.

2. The use as claimed in claim 1, for producing nucleic acid chips (DNA chips), producing protein chips, sorting genomic libraries, analyzing transcript libraries, measuring a variation in the activity of a functional protein, effecting antivirograms, and for protein screening or pharmaceutical screening.

3. The use as claimed in claim 1 or 2, characterized in that the maximum surface area of the bottom of each microcuvette is less than or equal to twice the smallest surface area of the biological object to be isolated.

4. The use as claimed in claim 3, characterized in that the surface area of said bottom is less than or equal to the smallest surface area of the biological object to be isolated.

5. The use as claimed in any one of the preceding claims, characterized in that the maximum surface area of the bottom of each microcuvette is between 1 μm² and 400 μm².

6. The use as claimed in claim 5, characterized in that the maximum surface area of the bottom of each microcuvette is between 1 and 50 μm².

7. The use as claimed in any one of the preceding claims, characterized in that the matrix of reaction microcuvettes of the device is surmounted, at least in part, by one or more layers of isolating materials and/or by an attached grid made of biocompatible plastic, so as to form a matrix of microreservoirs.

8. The use as claimed in claim 7, characterized in that the isolating materials are chosen from polyimides and resins.

9. The use as claimed in any one of the preceding claims, characterized in that the microreservoirs are between 5 and 500 μm in length and/or in width.

10. The use as claimed in any one of claims 7 to 9, characterized in that the device comprises at least two layers of isolating materials, and in that one of said layers is not an integral part of the device but is in the form of a removable, mounted component which covers at least in part said device.

11. The use as claimed in any one of the preceding claims, characterized in that one face of the first electrode integrated into the device constitutes the bottom of the microcuvettes.

12. The use as claimed in any one of claims 1 to 10, characterized in that the bottom of the microcuvettes consists of a layer made of glass, plastic or silicon.

13. The use as claimed in any one of the preceding claims, characterized in that the second electrode is integrated into the device and in that it is located in a plane apart from the bottom of the microcuvettes.

14. The use as claimed in any one of claims 1 to 12, characterized in that the second electrode is external to the device and in that it is joined to a cap or a lid.

15. The use as claimed in any one of the preceding claims, characterized in that a reagent capable of attaching the biological object to be isolated is attached to at least one part of the reception zone of the reaction microcuvettes.

16. The use as claimed in claim 15, taken in combination with any one of claims 11, 13 or 14, characterized in that the reagent is chosen from conducting copolymers to which are attached proteins, peptides or any molecules specific to the type of biological object to be attached.

17. The use as claimed in claim 16, characterized in that the conducting copolymers are chosen from polypyrroles.

18. The use as claimed in claim 16 or 17, characterized in that the reagent is a pyrrole-biotin-strepta-vidin-biotin-specific molecule copolymer.

19. The use as claimed in claim 15, taken in combination with any one of claims 12 to 14, characterized in that the reagent is chosen from polymers not specific for the type of biological object to be attached.

20. The use as claimed in claim 19, characterized in that said polymers are poly-L-lysine.

21. The use as claimed in claim 15, taken in combination with any one of claims 12 to 14, characterized in that the reagent is a protein or a peptide and in that said layer made of glass, plastic or silicon is covered with a layer of silane modified by —NHS or aldehyde functions to which said reagent is attached.

22. The use as claimed in any one of the preceding claims, characterized in that the device is equipped with a closing means.

23. The use as claimed in any one of the preceding claims, characterized in that the biological objects to be isolated are prokaryotic or eukaryotic cells, viruses, liposomes and microalgae.

24. The use as claimed in any one of the preceding claims, characterized in that the biological objects are chosen from bacteria derived from a library of genomic sequences.

25. A method for separating and/or isolating biological objects for obtaining nucleic acid chips or protein chips or for detecting functional proteins, characterized in that it comprises at least the following steps a) and b):
a) bringing at least one miniature device for separating and/or isolating biological objects, comprising at least one first electrode integrated into the device and at least one second electrode integrated into or external to the device, consisting of a structure provided with a matrix of reaction microcuvettes, each microcuvette comprising a bottom devoid of holes and constituting a reception zone, the maximum surface area of said bottom of each microcuvette being defined so as to isolate a single biological object, said structure being connected to a feed circuit so as to create a potential difference between said first electrode and said second electrode, into contact with a homogenized solution of biological objects, so as to allow the attachment of said biological objects at the bottom of the microcuvettes, on the reception zones, at a rate of at most one biological object per microcuvette,
b) removing the unattached biological objects so as to obtain a miniature device on which the biological objects to be isolated are immobilized.

26. The method as claimed in claim 25, characterized in that the biological objects are attached via an electric field.

27. The method as claimed in claim 25, characterized in that the biological objects are attached via a reagent attached to at least one part of the bottom of the reaction microcuvettes.

28. The method as claimed in any one of claims 25 to 27, characterized in that it comprises a step which is a step preliminary to step a), consisting in transforming the biological objects with a cloning vector bearing a gene which encodes a specific protein which will be present at the surface of the biological objects such that the attachment of the biological objects will take place via an interaction between this protein and a reagent specific for said protein, and/or by cloning a DNA fragment into a vector, said fragment preventing the expression of a gene encoding a protein which is toxic for the recombinant biological object.

29. The method as claimed in any one of claims 25 to 28, characterized in that a device comprising microreservoirs is used and in that it comprises a step during which the genetic material of the biological objects is released into said microreservoirs.

30. The method as claimed in any one of claims 25 to 28, characterized in that a device comprising microreservoirs and an internal or external second electrode suitable for permeabilization of the attached biological objects is used.

31. The method as claimed in any one of claims 25 to 30, characterized in that it is used for obtaining nucleic acid chips and in that it comprises a step during which the genetic material of the isolated objects is amplified so as to obtain amplified sequences.

32. The method as claimed in claim 31, characterized in that the amplified sequences are attached by electropolymerization to a second electrode of the device used.

33. The method as claimed in any one of claims 25 to 30, characterized in that it is used for obtaining protein chips or for detecting functional proteins, and in that it comprises, after step b), a step for transcription and translation of the genetic material of the isolated biological objects into proteins.

34. The method as claimed in claim 33, characterized in that it comprises a step of attachment of said proteins to a second electrode of the device used.

35. The method as claimed in claim 33 or 34, characterized in that it is used for detecting functional proteins and in that it comprises, after the translation step, a step for revealing at least one property of the isolated proteins.

36. The method as claimed in claim 33 or 34, characterized in that it comprises a step for measuring the variation in the activity of said functional proteins, consisting in assaying the effect of various molecules on the activity of said functional proteins.

37. The method as claimed in any one of claims 33 to 35, characterized in that it is used for obtaining recombinant protein chips and in that it comprises, before step a), a step for transforming the biological objects with a cloning vector in order to enable the recombinant proteins to be labeled with a universal epitope.

38. A method for analyzing a transcript library, characterized in that it comprises at least steps a) and b) as defined in claim 25, and also a step for detecting a gene which has experienced a difference in expression.
